(19) United States
(12) Patent Application Publication
(10) Pub. No.: US 2026/0232480 A1
(43) Pub. Date: Aug. 13, 2026

(54) PH SENSING SKIN ADHESIVE INCLUDING MESOPOROUS PARTICLES

(71) Applicant: Hollister Incorporated, Libertyville, IL (US)

(72) Inventors: Sohaila Abdelhamid, Chicago, IL (US); Mallory Brodnik, Fairfax, VA (US); Riley Forst, Chicago, IL (US); Stephanie Nofz, Montreal (CA); Lingfeng Tang, Guangzhou (CN); Luke Teasley, Fairfax Station, VA (US); Mustafa Guler, Wilmette, IL (US); Terry Johnson, Chicago, IL (US); Adrian P. Defante, Chicago, IL (US); Abram D. Janis, Grayslake, IL (US)

(73) Assignee: Hollister Incorporated, Libertyville, IL (US)

(21) Appl. No.: 19/153,585

(22) PCT Filed: Mar. 7, 2024
(86) PCT No.: PCT/US2024/018780

§ 371 Date: Aug. 4, 2025

Related U.S. Application Data
(60) Provisional application No. 63/489,341, filed on Mar. 9, 2023.

(51) Int. Cl. A61F 5/443; A61L 24/00; A61L 24/08; C08L 5/08

(52) U.S. Cl. CPC A61F 5/443; A61L 24/0031; A61L 24/08; C08L 5/08; A61L 2400/14

(57) ABSTRACT

A skin adhesive or an ostomy skin barrier includes a pH indicator encapsulated in mesoporous particles. The skin adhesive or the ostomy skin barrier is configured to change color in response to a change in pH.

### BACKGROUND

The present disclosure relates to skin adhesives, and more particularly to pH sensing hydrogels for skin adhesives.

Hydrogels are biomaterials designed for medical applications, such as drug delivery, regenerative medicine, tissue adhesion and wound treatments. Hydrogels are typically mechanically weak and have relatively low elastic moduli, which have historically presented limitations in applications requiring higher mechanical properties, such as ostomy applications.

Ostomy appliances, such as an ostomy pouch, are used to collect stoma dejecta (fecal material and/or urine, also referred to as dejecta) for patients with a stoma created by a surgery (e.g., urostomy, colostomy and ileostomy). An ostomy appliance is typically attached to user's peristomal skin via an ostomy wafer or an ostomy faceplate including a skin barrier. A skin barrier is designed to securely adhere to user's skin and support an ostomy appliance, while absorbing moisture and liquid from the peristomal skin and facilitate peristomal skin health. Some ostomy patients also use an ostomy ring that is made using a skin barrier material to fill in uneven skin contours around the stoma and/or to create a flatter peristomal skin surface for improved attachment of an ostomy wafer.

When an ostomy wafer is not properly fitted to the user, a leakage can occur and allow stoma dejecta to come in contact with the skin surrounding the stoma. This can cause irritation and can lead to broken skin and infection as well as interference with the adhesion of a skin barrier to user's skin. Many known ostomy skin barriers are formed of hydrocolloid adhesives, which can break down when exposed to stoma dejecta. Further, due to the continuous and prolonged use of ostomy appliances, user's peristomal skin is always at risk of irritation and injuries.

The present disclosure provides improved skin adhesives and skin barriers for medical applications, such as ostomy applications.

### BRIEF SUMMARY

In one aspect, a skin adhesive may be configured to change color in response to a change in pH and may include a pH indicator encapsulated in mesoporous particles.

In another aspect, an ostomy appliance for securing and supporting an ostomy pouch to a user may include a skin barrier configured to change color in response to a change in pH and comprising a pH indicator encapsulated in mesoporous particles. The ostomy appliance may be an ostomy wafer attached to an ostomy pouch or an ostomy faceplate comprising a body-side coupling member configured to engage with a pouch-side coupling member of an ostomy pouch.

In an embodiment, the skin adhesive or the skin barrier may be formed from a hydrogel. For example, the skin adhesive or the skin barrier may be formed from a chitosan hydrogel adhesive comprising a polyacrylamide network, a sodium alginate network, and a chitosan network. The chitosan hydrogel adhesive may be formed from a mixture comprising sodium alginate, acrylamide (AAm), calcium sulphate (CaSO₄), ammonium persulfate (APS), N,N′-methylenebisacrylamide (MBAA), and tetramethylethylenediamine (TEMED). The mixture may be crosslinked via chemical means or physical means, such as light (e.g. UV or visible), temperature and/or mechanical stimulation. In another embodiment, the skin adhesive or the skin barrier may be formed from a hydrocolloid adhesive.

In any of the foregoing embodiments, the pH indicator may be a universal pH indicator configured to change color along a full range of pH from about 1 to about 14. The universal pH indicator may be formed from a mixture of Methyl Red, Phenolphthalein, Ethanol, Bromothymol Blue, and NaOH. In another embodiment, the pH indicator may be configured to change color along a range of pH from about 4 to about 10.

In an embodiment, the pH indicator may be encapsulated in mesoporous silica particles or mesoporous silica nanoparticles to form an MSP pH indicator system (“MSP” as used herein refers to both mesoporous silica particles and mesoporous silica nanoparticles). The MSP pH indicator system may be dispersed in the skin adhesive or the skin barrier. In some embodiments, the MSP pH indicator system may be configured such that the pH indicator remains encapsulated in MSP until triggered by a stimulus, such as a change of temperature or a change of pH.

In an embodiment, the MSP pH indicator system may be configured to release the pH indicator when a pH of the skin barrier changes upon exposure to stoma dejecta, wherein the skin barrier may be configured to change color upon release of the pH indicator.

In an embodiment, the skin barrier may be configured to indicate a pH level of stoma dejecta that comes in contact with the skin barrier. In such an embodiment, the color of the skin barrier may be compared to a pH color scale for determination of the pH level of the stoma dejecta. In an embodiment, the skin barrier may be configured to change color when exposed to stoma dejecta to indicate a leakage.

In yet another aspect, an ostomy ring may be formed from the skin adhesive or the skin barrier of any of the foregoing embodiments. The ostomy ring may be configured to work with an ostomy wafer or an ostomy faceplate to securely attach the ostomy wafer or the ostomy faceplate to a user.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1A is a schematic illustration of a prior art skin barrier formed from a hydrocolloid adhesive attached to user's skin;

FIG. 1B is a schematic illustration of a skin barrier formed from a chitosan hydrogel adhesive according to an embodiment;

FIG. 2 is schematic illustration of pH indicator encapsulated in a mesoporous particle and released therefrom triggered by a stimulus according to an embodiment;

FIG. 3 is a schematic cross sectional view of an ostomy appliance comprising an ostomy pouch and an ostomy wafer according to an embodiment;

FIG. 4 is a schematic cross sectional view of the ostomy wafer of FIG. 3; and

FIG. 5 is an illustration of an ostomy ring according to an embodiment.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words “top,” “bottom,” up,“ ”down,“ front,” “back,” and the like are used for purposes of illustration and as such, are not limiting. Depending on the context, the word “if” as used herein may be interpreted as “when” or “upon” or “in response to determining.”

Human dejecta contains various bio-physio-chemical markers that can provide insight into the health condition of a person. Studies have shown links between pH of urine or fecal matter and diseases. For example, a significantly increased correlation between mortality and bacteremia percentages has been shown in patients with an overly acidic or basic fecal matter. The pH of a healthy person's urine is 4.5 to 8.0. The pH of a healthy human's feces is typically about 6.6 and sits slightly acidic as a result of the fermentation of sugars and the production of fatty acids in the human digestive system. In ileostomy patients, the median pH is 7.0 in duodenum, 6.3 in the proximal region, and 7.3 in the distal part of the small intestine-slightly more basic than people without ileostomy procedures.

Research regarding pH of feces and the gastrointestinal tract has also illuminated the relationship between colorectal cancer and fecal pH. It has been shown that a high colonic pH can promote carcinogen creation from bile acids in turn leading to colorectal cancer initiating and progressing in intestinal environments of a higher pH. Population studies have shown that patients without colon cancer exhibit a fecal pH of 6.6±0.44 whereas patients with colon cancer have a fecal pH of 8.0±0.44 (with a p value <0.01).

Further, it is well documented that beneficial bacteria tend to prefer slightly acidic gut microbiomes whereas harmful bacteria prefer a more basic environment. The proton exchange that occurs in many biochemical reactions conducted by bacteria serve as a method with which they alter and control the pH of their environment. As such, the population of harmful bacteria grows as the population of beneficial bacteria decreases. Further, the gastrointestinal pH and the effect it has on the bacterial population can impact absorption of vitamins, electrolytes, and activities of digestive enzymes. Thus, it is desireable to monitor pH of person's dejecta.

Each ostomate's stoma and peristomal skin topography is unique. The complexity and variations in stoma and peristomal skin topographies among ostomates present great challenges in providing skin barrier appliances that properly fit user's peristomal topography. As such, ostomates often experience a leakage, which exposes the skin barrier and the peristomal skin to stoma dejecta. When expose to stoma dejecta, skin complications, such as irritant dermatitis and infections, can develop.

In an embodiment, a skin adhesive or a skin barrier may be configured to respond to a change in pH. For example, a skin barrier for an ostomy wafer or ostomy faceplate for attaching an ostomy pouch to a user may be configured to change color when exposed to stoma dejecta to detect a leakage. In response, a user may change the ostomy wafer or faceplate to prevent further propagation of the leakage. In an embodiment, the skin barrier may comprise a pH indicator configured to change color in response to a change in pH, for example upon exposure to stoma dejecta and indicacte a pH level of stoma dejecta.

In an embodiment, a skin adhesive or a skin barrier may be formed from a hydrocolloid adhesive comprising a pH indicator. In another embodiment, a skin adhesive or a skin barrier may be formed from a hydrogel comprising a pH indicator. The pH indicator may be a universal pH indicator configured to change color along the full pH range (pH 1-pH 14). In such an embodiment, a user may be provided with a pH color scale for comparing the color of the skin barrier or the skin adhesive to determine the pH level.

The universal pH indicator may be formed from a mixture of Methyl Red, Phenolphthalein, 95% Ethanol Solution, Bromothymol Blue, and 0.1 M NaOH.

In an embodiment, the pH indicator may be configured to change color only along a range of pH from about 4 to about 10 as detection of extreme pH levels may not be necessary when dealing with urine, fecal or dejecta pH.

Integrating a pH indicator may present challenges for some skin barriers and skin adhesives. For example, one or more ingredients of the skin barrier or skin adhesive may react with the pH indicator, resulting in unexpected alterations of properties of the skin barrir or skin adhesive. Further, some pH indicator may change color upon contact with user's skin and may stain clothing around the area. To solve such problems, in some embodiments, mesoporous particles may be used as a carrier for the pH indicator. The mesoporous particles may have a pore size of about 2 nm to about 50 nm.

In an embodiment, the pH indicator may be encapsulated in mesoporous particles. The mesoporous particles may be configured to provide good biocompatibility, good reactive surface, high pH indicator encapsulation efficiency and tunable pore size. In an embodiment, the pH indicator may be provided as a solution and encapsulated in the mesoporous particles by a coacervation process, such as suspension polymerization, emulsion polymerization, and the like. In another embodiment, the pH indicator solution may be encapsulated in the mesoporous particles by a physico-mechanical process, such as spray-drying, microfluidics, and layer-by-layer building technique.

In an embodiment, a skin adhesive or an ostomy skin barrier may be formed from a hydrocolloid adhesive comprising a pH indicator encapsulated in mesoporous particles. In another embodiment, an ostomy skin barrier or a skin adhesive may be formed from a hydrogel comprising a pH indicator encapsulated in mesoporous particles.

Hydrogels are water-insoluble, three-dimensional network of polymer chains capable of holding large amounts of water. In general, hydrogels can fall into two major categories: chemical hydrogels and physical hydrogels. Chemical hydrogels have covalent cross-linking bonds, whereas physical hydrogels have non-covalent bonds. Hydrogels may be designed for use in the human body and can provide excellent biocompatibility. Hydrogels have a significantly lower elastic modulus when compared to hydrocolloid adhesives and can be configured to have a similar elastic modulus to that of abdominal skin to improve user's comfort. However, hydrogels typically have relatively weak mechanical properties.

Double network hydrogels (DN hydrogels) comprising at least two different polymer networks, such as those formed from poly(2-acrylamido-2-methylpropanesulfonic acid) (PAMPS) and polyacrylamide (PAAm), can be configured to have improved mechanical properties including fracture toughness of 102-103 J/m², fracture tensile stress of 1-10 MPa, and fracture tensile strain of 1000-2000%. Such improvements in mechanical properties of the DN hydrogels may be attributed to their unique network structure as well as the entanglements within the structure. Hybrid DN hydrogels include both physically crosslinked components and chemically crosslinked components, wherein the first network is formed by physically crosslinked gels and the second network is chemically crosslinked. The associations between the networks may be non-covalent interactions such as van der Waals interactions, hydrogen bonds, and electrostatic association, depending on the intrinsic properties of the polymers.

In an embodiment, an ostomy skin barrier or a skin adhesive may be formed from a DN hydrogel (the term “DN hydrogel” herein broadly includes both DN hydrogels and hybrid DN hydrogels) comprising a pH indicator encapsulated in mesoporous particles. The skin barrier formed from a DN hydrogel may be configured to provide improved biocompatibility, non-volatility, and superior flexibility (low elastic modulus) when compared to skin barriers formed from hydrocolloid adhesives. Further, the DN hydrogel may be configured to provide adhesive properties suitable for supporting ostomy appliances and have antimicrobial properties to reduce the risk of peristomal skin infections and complications.

In an embodiment, the DN hydrogel may be a hybrid DN hydrogel comprising a physically crosslinked alginate component and a chemically crosslinked acrylamide (AAm) component. The hybrid DN hydrogel may also comprise chitosan (also referred to herein as “chitosan hydrogel adhesive”). In an embodiment, the chitosn hydrogel adhesive may comprise a polyacrylamide network, sodium alginate network, and chitosan network.

In an embodiment, the chitosan hydrogel adhesive may be formed from a mixture of sodium alginate, AAm, CaSO4, ammonium persulfate (APS), N,N′-methylenebisacrylamide (MBAA), and tetramethylethylenediamine (TEMED). A sample of the chitosan hydrogel adhesive was formed via the following synthesis steps: 1) 1.028 g of sodium alginate and 5.3575 g of AAm were dissolved in 50 ml of diH2O for 24 hours until the sodium alginate was dissolved; 2) 17.5 ml of the alginate/AAm solution was mixed with 600 μl of MBAA (0.2 g per 100 ml), 100 μl of APS (0.75 M), 400 μl of CaSO4 (0.27 M) and 10 μl of TEMED; and 3) the solution was quickly poured into a mold to prevent quick gelation of the alginate with the ionic crosslinker, and the gel was cured under UV light.

In an embodiment, an ostomy skin barrier may be formed from the chitosan hydrogel adhesive comprising a pH indicator encapsulated in mesoporous particles and configured to change color in response to a change in pH. For example, the skin barrier may change color when exposed to stoma dejecta and indicate a pH level of the stoma dejecta. The pH indicator encapsulated in mesoporous particles may be configured to provide a distinct visual cue when exposed to stoma dejecta to function as a leakage indicator. In response to the visual cue, such as a color change of the skin barrier, a user may change the skin barrier to prevent propagation of leakage and further exposure to stoma dejecta and avoid skin complications caused by irritants in the dejecta.

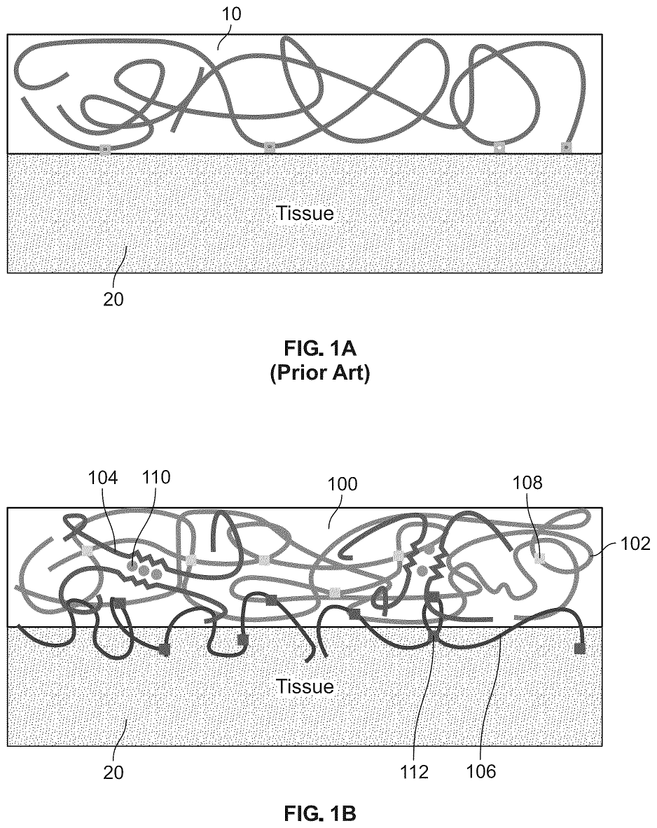

In such an embodiment, the chitosan network in the chitosan hydrogel adhesive may function as an adhesion enhancer to provide a strong and flexible topological adhesion between user's skin and the chitosan hydrogel adhesive. FIG. 1A is a schematic illustration of a prior art skin barrier 10 formed from a hydrocolloid adhesive attached to user's skin 20. FIG. 1B is a schematic illustration of a skin barrier 100 formed from a chitosan hydrogel adhesive attached to user's skin 20 according to an embodiment. The skin barrier 100 may be formed from a chitosn hydrogel adhesive comprising polyacrylamide network 102, sodium alginate network 104, chitosan network 106, polyacrylamide crosslinker 108, ionic crosslinker (calcium ion) 110, and chitosan crosslinker 112. As shown in FIG. 1B, the chitosan network 106 may interact with user's skin 20 and function as an adhesion enhancer to provide the adhesive properties sufficient for supporting an ostomy appliance, such as an ostomy pouch.

In an embodiment, the DN hydrogel comprising a pH indicator encapsulated in mesoporous particles may be formed by adding the pH indicator mesoporous particles to an alginate/AAm solution prepared by dissolving sodium alginate and AAm in diH₂O, and mixing MBAA, APS, CaSO4, and TEMED into the alginate/AAm solution to form a gel, and curing the gel using UV light.

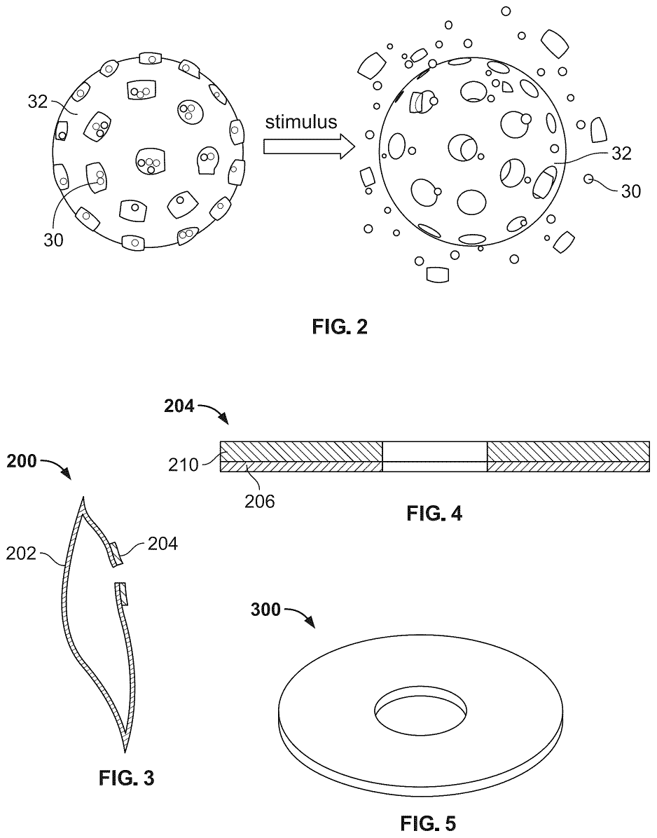

In an embodiment, an ostomy skin barrier or a skin adhesive may be formed from a chitosan hydrogel adhesive comprising a pH indicator encapsulated in mesoporous silica nanoparticles (“MSP pH indicator system”). The MSP pH indicator system may be configured such that the pH indicator may remain encapsulated in MSP until triggered by a stimulus, such as pH, ultrasound and temperature. In an embodiment, the MSP pH indicator system may be configured to release the pH indicator when the temperature of the skin barrier increases upon exposure to stoma dejecta. In another embodiment, the MSP pH indicator system may be configured to release the pH indicator when the pH of the skin barrier changes upon exposure to stoma dejecta from a leakage. In such embodiments, the release of the pH indicator may change the color of the skin barrier to indicate a leakage. The MSP pH indicator system may include a universal pH indicator, wherein a user may compare the color of the skin barrier upon exposure to stoma dejecta against a pH color scale to determine the pH of the stoma dejecta. FIG. 2 is a schematic illustration of a pH indicator 30 encapsulated in a mesoporous particle 32 and released therefrom triggered by a stimulus, such as a change of pH.

In some embodiments, a skin adhesive or an ostomy skin barrier may be formed from a DN hydrogel configured to swell in response to a change in pH and comprising a MSP pH indicator system. For example, the ostomy skin barrier may be formed from an acidic DN hydrogel that swells when exposed to stoma dejecta having a basic pH. In another example, the ostomy skin barrier may be formed from a basic DN hydrogel that swells when exposed to stoma dejecta having an acidic pH. In yet another example, the ostomy skin barrier may be formed from an amphiphilic hydrogel that swells when exposed to dejecta having an acidic pH or a basic pH.

In any of the foregoing embodiments, the DN hydrogel may be configured to have viscoelastic properties similar to that of skin to provide a skin barrier that bends and folds with user's abdominal skin rather than tug and pull at it to improve user's comfort. For example, the DN hydrogel may be configured to have an elastic modulus of about 10 kPa to about 1 MPa. Such DN hydrogel may conform to user's peristomal topography better and provide an improved adhesive seal when compared to hydrocolloid adhesives.

FIG. 3 is a schematic cross sectional view of an ostomy appliance 200 comprising an ostomy pouch 202 and an ostomy wafer 204 according to an embodiment. FIG. 4 is a schematic cross sectional view of the ostomy wafer 204. In an embodiment, the ostomy wafer 204 may comprise a backing layer 206 and a skin barrier 210 formed from the DN hydrogel of any of the foreging embodiments. FIG. 5 is an illustration of an ostomy ring 300 fromed from the DN hydrogel of any of the foregoing embodiments.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A skin adhesive comprising a pH indicator encapsulated in mesoporous particles, wherein the skin adhesive is configured to change color in response to a change in pH.

2. The skin adhesive of claim 1, wherein the skin adhesive is formed from a hydrogel.

3. The skin adhesive of claim 1, wherein the skin adhesive is formed from a hydrocolloid adhesive.

4. The skin adhesive of claim 1, wherein the skin adhesive is formed from a chitosan hydrogel adhesive comprising a polyacrylamide network, a sodium alginate network, and a chitosan network.

5. The skin adhesive of claim 4, wherein the chitosan hydrogel adhesive is formed from a mixture comprising sodium alginate, acrylamide (AAm), calcium sulphate (CaSO4), ammonium persulfate (APS), N,N′-methylenebisacrylamide (MBAA), and tetramethylethylenediamine (TEMED).

6. The skin adhesive of claim 1, wherein the pH indicator is a universal pH indicator configured to change color along a full range of pH from about 1 to about 14, wherein the universal pH indicator is formed from a mixture of Methyl Red, Phenolphthalein, Ethanol, Bromothymol Blue, and NaOH.

7. The skin adhesive of claim 1, wherein the pH indicator is configured to change color along a range of pH from about 4 to about 10.

8. The skin adhesive of claim 1, wherein the pH indicator is encapsulated in mesoporous silica particles to form a MSP pH indicator system, wherein the MSP pH indicator system is dispersed in the skin adhesive.

9. The skin adhesive of claim 8, wherein the MSP pH indicator system is configured such that the pH indicator remains encapsulated in MSP until triggered by a stimulus.

10. The skin adhesive of claim 9, wherein the stimulus is a change of temperature or a change of pH.

11. An ostomy appliance configured to secure and support an ostomy pouch to a user comprising a skin barrier, wherein the skin barrier is formed from the skin adhesive of claim 1.

12. The ostomy appliance of claim 11, wherein the ostomy appliance is an ostomy wafer attached to an ostomy pouch or an ostomy faceplate comprising a body-side coupling member configured to engage with a pouch-side coupling member of an ostomy pouch.

13-18. (canceled)

19. The ostomy appliance of claim 11, wherein the skin barrier is configured to indicate a pH level of stoma dejecta that comes in contact with the skin barrier, wherein a color of the skin barrier is compared to a pH color scale for determination of a pH level of the stoma dejecta.

20. The ostomy appliance of claim 11, wherein the skin barrier is configured to change color when exposed to stoma dejecta to indicate a leakage.

21. The ostomy appliance of claim 11, wherein the pH indicator is encapsulated in mesoporous silica particles to form a MSP pH indicator system, wherein the MSP pH indicator system is dispersed in the skin adhesive.

22. The ostomy appliance of claim 21, wherein the MSP pH indicator system is configured such that the pH indicator remains encapsulated in MSP until triggered by a stimulus.

23. The ostomy appliance of claim 22, wherein the stimulus is a change of temperature or a change of pH.

24. The ostomy appliance of claim 22, wherein the MSP pH indicator system is configured to release the pH indicator when a pH of the skin barrier changes upon exposure to stoma dejecta, wherein the skin barrier is configured to change color upon release of the pH indicator.

25. An ostomy ring formed from the skin adhesive of claim 1, wherein the ostomy ring is configured to work with an ostomy wafer or an ostomy faceplate to securely attach the ostomy wafer or the ostomy faceplate to a user.
